(19) United States
(12) Patent Application Publication
(10) Pub. No.: US 2014/0290651 A1
(43) Pub. Date: Oct. 2, 2014

(54) CRUDE NEON WITH NITROGEN AND OXYGEN AS A HYPERBARIC INTERVENTION BREATHING MIXTURE

(71) Applicant: Hamilton Research, Ltd., Tarrytown, NY (US)

(72) Inventors: David J. Kenyon, Morristown, NJ (US); Glenn Butler, Tarrytown, NY (US)

(21) Appl. No.: 14/229,747

(22) Filed: Mar. 28, 2014

Related U.S. Application Data
(60) Provisional application No. 61/806,390, filed on Mar. 28, 2013.

(51) Int. Cl. A61G 10/02; A61M 16/12

(52) U.S. Cl. CPC A61G 10/026; A61M 16/12; A61M 2202/025; A61M 2202/0266; A61M 2202/0208
USPC 128/203.12; 96/134

(57) ABSTRACT

Embodiments of the present invention provide systems, methods and apparatus for to using crude neon with oxygen and nitrogen as a hyperbaric intervention breathing mixture. Embodiments include providing a work environment under pressure; performing work operations within the pressurized work environment; and providing a breathing mixture created from crude neon and oxygen as a hyperbaric intervention breathing gas. Numerous additional aspects are disclosed.

### RELATED APPLICATIONS

The present application claims priority to U.S. Provisional Patent Application Ser. No. 61/806,390, filed Mar. 28, 2013 and entitled “CRUDE NEON WITH NITROGEN AND OXYGEN AS A HYPERBARIC INTERVENTION BREATHING MIXTURE”, (Attorney Docket No. LS-011/L), which is hereby incorporated herein by reference in its entirety for all purposes.

### FIELD

The present invention relates generally to hyperbaric intervention, and more particularly to using crude neon with oxygen and nitrogen as a hyperbaric intervention breathing mixture.

### BACKGROUND

A breathing mixture or breathing gas is a combination of gaseous chemical elements and compounds used for respiration. The essential component for any breathing gas is a partial pressure of oxygen (ppO₂) of between roughly 0.16 and 1.60 bar at the ambient pressure. The oxygen is usually the only metabolically active component unless the gas is an anesthetic mixture. Some of the oxygen in a breathing mixture is consumed by the metabolic processes, and the inert components are unchanged, and serve mainly to dilute the oxygen to an appropriate concentration, and are therefore also known as diluent gases.

Air is the most common and only natural breathing mixture. Other gases, either pure gases or mixtures of gases, are used in breathing equipment and enclosed habitats such as SCUBA equipment, surface supplied diving equipment, recompression chambers, submarines, space suits, spacecraft, medical life support and first aid equipment, high-altitude mountaineering and anesthetic machines.

Most breathing gases are a mixture of oxygen and one or more inert gases. Other breathing mixtures have been developed to improve on the performance of air by reducing the risk of decompression sickness, reducing the duration of decompression stops, reducing nitrogen narcosis or allowing safer deep diving. A safe breathing mixture for hyperbaric use has three essential features: (1) it contains sufficient oxygen to support life, consciousness and work rate of the breather; (2) it does not contain harmful gases such as carbon monoxide and carbon dioxide which are common poisons which can contaminate breathing gases; and (3) it does not become toxic when being breathed at the intended pressures such as when deep underwater. Oxygen and nitrogen are examples of gases that become toxic under pressure.

The techniques used to fill diving cylinders with gases other than air are called gas blending. There are several different breathing mixes that are used for applications such as diving. These include: Air, Pure Oxygen, Nitrox, Trimix, Heliox, Heliair, Hydreliox, Hydrox, and Neox. Air is a mixture of 21% oxygen, 78% nitrogen, and approximately 1% other trace gases, primarily argon; to simplify calculations this last 1% is usually treated as if it were nitrogen. Being cheap and simple to use, it is the most common diving gas. As its nitrogen component causes nitrogen narcosis, it is considered to have a safe depth limit of about 40 meters for most divers, although the maximum operating depth of air is 66.2 meters.

Pure oxygen is mainly used to speed the shallow decompression stops at the end of a military, commercial, or technical dive and is only safe down to a depth of 6 meters (maximum operating depth) before oxygen toxicity becomes a problem. Pure oxygen was frequently used in military rebreathers.

Nitrox is a mixture of oxygen and air, and generally refers to mixtures which are more than 21% oxygen. Nitrox can be used as a tool to accelerate in-water decompression stops or to decrease the risk of decompression sickness and thus prolong a dive. However, Nitrox has a shallower maximum operating depth than conventional air.

Trimix is a mixture of oxygen, nitrogen and helium and is often used at depth in technical diving and commercial diving instead of air to reduce nitrogen narcosis and to avoid the dangers of oxygen toxicity. Further details regarding the use of trimix can be found in R. Takashima et al., “Use of Trimix breathing in deep caisson work for the construction of the Meiko West Bridge,” Undersea Hyperbaric Med. 23(suppl.), 34 (1996), which is hereby incorporated herein by reference. Heliox is a mixture of oxygen and helium and is often used in the deep phase of a commercial deep dive to eliminate nitrogen narcosis.

Heliair is a form of trimix that is blended from helium and air without using pure oxygen. Heliair has a 21:79 ratio of oxygen to nitrogen and the balance of the mix is helium. Hydreliox is a mixture of oxygen, helium, and hydrogen and is used for dives below 130 meters in commercial diving. Hydrox, a gas mixture of hydrogen and oxygen, is used as a breathing gas in very deep diving. Neox (also called neonox) is a mixture of oxygen and neon sometimes employed in deep commercial diving. However, Neox is rarely used due to the high cost of neon.

Oxygen (O₂) must be present in every breathing mixture. This is because it is essential to the human body's metabolic process, which sustains life. The human body cannot store oxygen for later use as it does with food. If the body is deprived of oxygen for more than a few minutes, unconsciousness and death result. The tissues and organs within the body (notably the heart and brain) are damaged if deprived of oxygen for much longer than four minutes.

Filling a diving cylinder with pure oxygen costs around five times more than filling it with compressed air. As oxygen supports combustion and causes rust in diving cylinders, it has to be handled with caution when gas blending. Oxygen has historically been obtained by fractional distillation of liquid air, but is increasingly obtained by non-cryogenic technologies such as pressure swing adsorption (PSA) and vacuum-pressure swing adsorption (VPSA) technologies.

The fraction of the oxygen component of a breathing gas mixture is sometimes used when naming the mix. Hypoxic mixes, strictly, contain less than 21% oxygen, although often a boundary of 16% is used, and are designed only to be breathed at depth as a “bottom gas” where the higher pressure increases the partial pressure of oxygen to a safe level. Trimix, Heliox and Heliair are gas blends commonly used for hypoxic mixes and are used in professional and technical diving gas deep breathing mixtures.

Normoxic mixes have the same proportion of oxygen as air, 21%. The maximum operating depth of a normoxic mix could be as shallow as 47 meters. Trimix with between 17% and 21% oxygen is often described as normoxic because it contains a high enough proportion of oxygen to be safe to breathe at the surface.

Hyperoxic mixes have more than 21% oxygen. Enriched Air Nitrox (EANx) is a typical hyperoxic breathing gas. Hyperoxic mixtures, when compared to air, cause oxygen toxicity at shallower depths but can be used to shorten decompression stops by drawing dissolved inert gases out of the body more quickly.

The fraction of the oxygen determines the greatest depth at which the mixture can safely be used to avoid oxygen toxicity. This depth is called the maximum operating depth. The concentration of oxygen in a gas mix depends on the fraction and the pressure of the mixture. It is expressed by the partial pressure of oxygen (ppO₂). The partial pressure of any component gas in a mixture is calculated as:

partial pressure=total absolute pressure x volume fraction of gas component

For the oxygen component,

ppO₂=P×FO₂

where ppO₂ represents the partial pressure of oxygen, P represents the total pressure, and FO₂ represents the volume fraction of oxygen content.

The minimum safe partial pressure of oxygen in a breathing gas is commonly held to be 16 kPa (0.16 bar). Below this partial pressure the diver may be at risk of unconsciousness and death due to hypoxia, depending on factors including individual physiology and level of exertion. When a hypoxic mix is breathed in shallow water it may not have a high enough ppO₂ to keep the diver conscious. For this reason normoxic or hyperoxic “travel gases” are used at medium depth between the “bottom” and “decompression” phases of the dive.

The maximum safe ppO₂ in a breathing gas depends on exposure time, the level of exercise and the security of the breathing equipment being used. It is typically between 100 kPa (1 bar) and 160 kPa (1.6 bar); for dives of less than three hours it is commonly considered to be 140 kPa (1.4 bar), although the U.S. Navy has been known to authorize dives with a ppO₂ of as much as 180 kPa (1.8 bar). At high ppO₂ or longer exposures, the diver risks oxygen toxicity which may result in a seizure. Each breathing gas has a maximum operating depth that is determined by its oxygen content. For therapeutic recompression and hyperbaric oxygen therapy partial pressures of 2.8 bar are commonly used in the chamber, but there is no risk of drowning if the occupant loses consciousness. Oxygen analyzers are used to measure the ppO₂ in the gas mix.

Nitrogen (N₂) is a diatomic gas and the main component of air, the cheapest and most common breathing gas used for diving. It causes nitrogen narcosis in the diver, so its use is limited to shallower dives. Nitrogen can cause decompression sickness.

Equivalent air depth is used to estimate the decompression requirements of a nitrox (oxygen/nitrogen) mixture. Equivalent narcotic depth is used to estimate the narcotic potency of trimix (oxygen/helium/nitrogen mixture). Many divers find that the level of narcosis caused by a 30 meter dive, while breathing air, is a comfortable maximum. Nitrogen in a breathing mixture is almost always obtained by adding air to the mix.

Helium (He) is an inert gas that is less narcotic than nitrogen at equivalent pressure (in fact there is no evidence for any narcosis from helium at all), so it is more suitable for deeper dives than nitrogen. Helium is equally able to cause decompression sickness. At high pressures, helium also causes High Pressure Nervous Syndrome, which is a central nervous system irritation affliction which is in some ways opposite to narcosis. The use of helium typically costs ten times more than an equivalent amount of air.

Helium is not very suitable for dry suit inflation owing to its poor thermal insulation properties—helium is a very good conductor of heat (compared to air which is a rather poor, making it more of an insulator). Helium's low molecular weight (monatomic MW=4, compared with diatomic nitrogen MW=28) increases the timbre of the breather's voice, which may impede communication. This is because the speed of sound is faster in a lower molecular weight gas, which increases the resonance frequency of the vocal cords. Helium leaks from damaged or faulty valves more readily than other gases because atoms of helium are smaller allowing them to pass through smaller gaps in seals. Helium is found in significant amounts only in natural gas, from which it is extracted at low temperatures by fractional distillation.

Neon (Ne) is an inert gas sometimes used in deep commercial diving but is very expensive Like helium, it is less narcotic than nitrogen, but unlike helium, it does not distort the diver's voice. Neon makes up approximately 0.0018 percent of the Earth's atmosphere. Although neon is relatively rare on earth, neon is the fifth most abundant element in the universe. To illustrate the amount of neon in the atmosphere, consider that if all the neon was gathered from the rooms in a typical new home, there would be about 10 liters (i.e., 2 gallons) of neon gas. Neon forms in stars with a mass of eight or more times the mass of earth's suns and neon has no stable compounds.

Neon is a colorless, noble gas with an atomic weight of 20.180, a melting point of −248.57 C, a boiling point of −246.0 C, it has 10 electrons, 10 protons, 10 neutrons, the electron shells are 2.8; and neon's density at 20 C is 0.0009 g/cm³. Pure neon costs more than 32 times the cost of helium. Each breath of a breathing mixture made from pure neon and oxygen at 400 fsw (13.1 Atm) costs approximately USD $20. Whereas helium can be found in abundance together with natural gas as a by-product of radioactive decay; neon can only be extracted from the air.

Hydrogen (H₂) has been used in deep diving gas mixes but is very explosive when mixed with more than about 4 to 5% oxygen (such as the oxygen found in breathing gas). This limits use of hydrogen to deep dives and imposes complicated protocols to ensure that oxygen is cleared from the lungs, the blood stream and the breathing equipment before breathing hydrogen starts. Like helium, it raises the timbre of the diver's voice. The hydrogen-oxygen mix when used as a diving gas is sometimes referred to as hydrox. Mixtures containing both hydrogen and helium as diluents are termed hydreliox.

Thus, what is needed is a breathing mixture that is safe and effective, cost efficient to manufacture, relatively easy to mix, does not have the drawbacks of existing breathing mixtures, and is suitable for many applications.

### SUMMARY

Embodiments of the present invention provide methods of using crude neon with oxygen and nitrogen as a hyperbaric intervention breathing mixture. The methods include providing a work environment under pressure; performing work operations within the pressurized work environment; and providing a breathing mixture created from crude neon and oxygen as a hyperbaric intervention breathing gas.

In other embodiments, the present invention provides systems for producing crude neon with oxygen as a hyperbaric intervention breathing mixture. The systems include an air separation plant; a hydrogen removal portion configured to receive a first fluid stream from the air separation plant; and an adsorbent bed portion configured to receive a second fluid stream from the hydrogen removal portion and further adapted to provide crude neon for use in a hyperbaric intervention breathing mixture.

In still other embodiments, the present invention provides methods of using crude neon with oxygen and nitrogen as a hyperbaric intervention breathing mixture for decompression. The methods include providing a decompression chamber under pressure; decompressing a user within the pressurized decompression chamber; and providing a breathing mixture created from crude neon and oxygen as a hyperbaric intervention breathing gas.

Other features and aspects of the invention will become more fully apparent from the following detailed description of example embodiments, the appended claims, and the accompanying drawings.

### BRIEF DESCRIPTION OF THE DRAWINGS

FIG. 1 is a schematic diagram illustrating an example crude neon production system according to embodiments of the present invention.

FIG. 2 is an example experimental dive schedule for testing breathing mixtures according to embodiments of the present invention.

FIG. 3 is a table of example test data summarizing decompression results after various times at different pressures according to embodiments of the present invention.

FIG. 4 is an example graph plotting the effect of increasing pressure on ventilation for different gases according to embodiments of the present invention.

FIG. 5 is an example graph plotting respiratory resistance at increased gas density for different gases according to embodiments of the present invention.

FIG. 6A is a table listing crude neon breathing mixtures including ranges of component percentages according to embodiments of the present invention.

FIG. 6B is a table listing time limits for working levels of oxygen partial pressure according to embodiments of the present invention.

FIG. 6C is a table listing time limits for resting levels of oxygen partial pressure according to embodiments of the present invention.

FIG. 6D is a table listing time limits for chamber levels of oxygen partial pressure according to embodiments of the present invention.

FIG. 7 is a flowchart depicting an example method according to embodiments of the present invention.

### DETAILED DESCRIPTION

Embodiments of the present invention relate to the safe exposure and subsequent decompression of humans and animals to hyperbaric pressures beyond the limits of air. This includes divers, caisson workers and tunnel boring machine operators who are regularly exposed to pressure greater than one atmosphere. In some embodiments of the present invention, crude neon, which may be produced relatively inexpensively as a by-product of the manufacture of cryogenic atmospheric gases, can be used in combination with nitrogen (N₂) and Oxygen (O₂) as an alternative to pure helium and oxygen mixtures such as HeliOx for intermediate pressure ranges in hyperbaric interventions. According to embodiments of the present invention, crude neon can be produced in two types: as crude neon 50 including 50% to 60% Ne, 20% to 30% N2 with the balance being helium, and as crude neon 75 including 70% to 80% Ne with the balance being helium. Thus, crude neon has several advantages over other gases. Specifically, crude neon has lower costs compared with helium production when the process is employed at a large commercial scale; crude neon is warmer for divers immersed in the lock-out gas; speech is more intelligible because the crude neon does not distort sound like helium does; and crude neon can be made available worldwide with a simple conversion of existing liquefaction plants.

As will be described in more detail below, crude neon can be cost-effectively obtained, for example, as a by-product of the manufacture of cryogenic atmospheric gases. This by-product is conventionally recycled for further processing to isolate pure neon or simply wasted by venting it to the atmosphere. Crude neon is the uncondensed fraction remaining after oxygen, argon and nitrogen have been recovered from air. Neon naturally occurs in the atmosphere at a level of about 20 ppm (0.002%). By using successive pressure swing sieves systems (e.g., as described in U.S. Pat. No. 5,100,446 to Michael W. Wisz issued Mar. 31, 1992, which is hereby incorporated herein by reference) and after removal of CO₂, water vapor, and the reclamation of liquid oxygen, argon and some nitrogen, the remaining uncondensed tail stream left is a mixture with a concentration of approximately 30% to 50% neon, approximately 20% to 30% helium, approximately 30% to 50% nitrogen, and a trace amount of oxygen. This tail stream gas is referred to as crude neon 50. Additional sieve separation can be used to further refine the crude neon 50 mixture to a mixture of approximately 70% to 80% neon and approximately 20% to 30% helium. This further refinement of crude neon 50, which includes the removal of both nitrogen and oxygen, is referred to as crude neon 75. One compelling reason to consider the use of crude neon 50 and 75 is that these mixtures can be prepared at any location in unlimited quantity without having to comply with legal and safety regulations that are associated with other gases such as, for example, helium or oxygen for medical applications.

Crude neon is mentioned as a by-product and an intermediate product for making pure neon in previously incorporated U.S. Pat. No. 5,100,446. The reference describes a crude neon production system wherein a small neon-containing stream is taken from a cryogenic air separation plant and processed in a neon column and in a non-cryogenic pressure swing adsorption system to produce crude neon for further processing to make pure neon and wherein tail gas with some neon from the pressure swing adsorption is recycled back into the air separation plant.

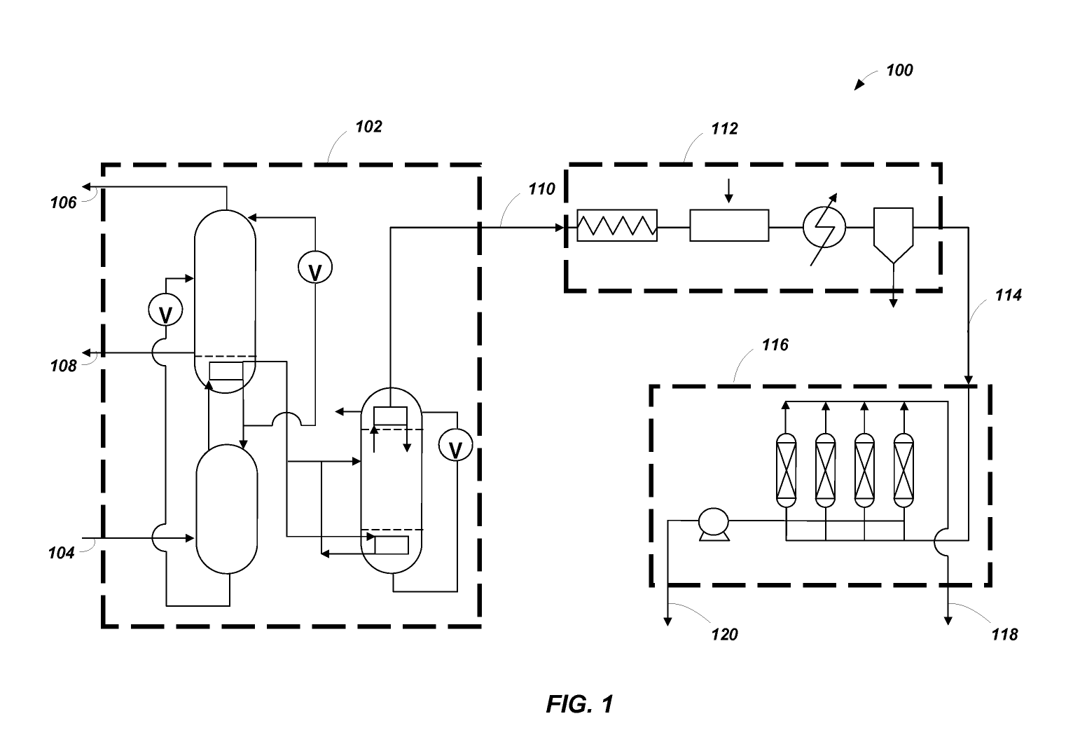

Turning now to FIG. 1, a schematic representation of a crude neon production system 100 according to the present invention is depicted. The system 100 includes a conventional cryogenic air separation plant 102 that receives a stream of feed air 104 which has been compressed, cleaned of high boiling impurities such as water and carbon dioxide, and cooled. The equipment to produce the feed air 104 including the feed air compressor, prepurifier and heat exchangers which normally comprise the warm end portion of a processing plant are not shown. In the embodiment illustrated in the FIG. 1, the air separation plant 102 is a double column system comprising a higher pressure column and a lower pressure column in heat exchange relation at a main condenser. The feed air 104 is provided into the higher pressure column which is operating at a pressure generally within the range of from 70 to 150 pounds per square inch absolute (psia). Within the column the feed air is separated by cryogenic rectification into nitrogen-richer and oxygen-richer components. The nitrogen-richer component is passed as vapor into a main condenser wherein it is condensed by indirect heat exchanger with reboiling column bottoms. Resulting condensed nitrogen-richer component is returned to the column as reflux.

The oxygen-richer component is passed from the column as a liquid stream into the lower pressure column which is operating at a pressure less than that of the higher pressure column and generally within the range of from 15 to 25 psia. In addition, a portion of the stream is expanded and introduced into the lower pressure column. Within the lower pressure column, the feeds are separated into a nitrogen stream 106 and an oxygen stream 108 which are removed as separate streams. Either or both of these streams 106, 108 can be recovered as product.

Because neon has a boiling point which is significantly less than that of nitrogen, the neon in the feed air 104 concentrates at the top of the higher pressure column and is passed into the main condenser. As the vapor condenses in the main condenser, the remaining uncondensed vapor at the top part of the main condenser grows progressively richer in neon, along with other low boiling components of the air such as hydrogen and helium. A vapor stream containing neon is taken from the main condenser and passed as feed into a neon column at a flow rate within the range of from 0.1 to 1.0 percent of the flow rate of the feed air 104 into the air separation plant 102. The main condenser can be a differential type condenser. The neon-containing vapor stream has a neon concentration which exceeds that of the feed air 104 and generally the neon concentration will be within the range of from 0.2 to 2.0 percent. The neon-containing vapor stream is then divided into two portions, one portion provided directly into the neon column and the other portion passed into a bottom reboiler. In the reboiler, the other portion is cooled by indirect heat exchange with boiling neon column bottoms so as to provide vapor boilup for the neon column. The resulting stream is recombined with the neon-containing vapor stream from the main condenser and passed back into the neon column.

Within the neon column, the neon-containing vapor stream is separated by cryogenic rectification into a vapor enriched in neon and a liquid enriched in nitrogen. The vapor is passed into a top reflux condenser wherein it is condensed and returned as reflux for the neon column. The liquid is provided from the bottom of the neon column and expanded into the boiling side of the reflux condenser where it is boiled to carry out the condensation of vapor. The resulting gaseous nitrogen is passed out from the neon column. A portion of the vapor does not condense in the top reflux condenser and in this vapor portion the neon is concentrated. Also concentrated in this vapor portion are low boiling components of air such as hydrogen and helium. The vapor portion is passed out from the top condenser as a neon-containing fluid stream 110 having a further reduced nitrogen concentration and an increased neon concentration. The nitrogen concentration of stream 110 will generally be within the range of from 10 to 30 percent and the neon concentration of stream 110 will generally be within the range of from 50 to 65 percent. The remainder of stream 110 includes primarily helium and hydrogen.

Stream 110 is next processed to remove hydrogen. An example hydrogen removal portion 112 of the crude neon production system 100 includes a heater to heat the stream 110 which is then provided into a catalytic reactor along with oxygen. Generally, the catalyst in catalytic reactor can be a palladium catalyst or the like. Within the catalytic reactor, the oxygen and hydrogen react in an exothermic reaction to form water. The resulting fluid is taken from the catalytic reactor, cooled through a cooler and passed through a separator wherein condensed water is removed. The resulting fluid stream 114 is then passed to an adsorbent bed portion 116 of the crude neon production system 100.

An adsorbent bed portion 116 useful with the present system 100 includes one or more beds with an adsorbent which adsorbs nitrogen over neon such as a molecular sieve (e.g., type 5A zeolite). The stream 114 is passed through the adsorbent bed portion 116 at an elevated pressure generally within the range of from 60 to 140 psia. At this elevated pressure the nitrogen is preferentially adsorbed over neon onto the bed resulting in the production of a crude neon 75 product containing substantially no nitrogen. Of course, some neon is also adsorbed by the adsorbent bed portion 116. The crude neon 75 product has a neon concentration within the range of from 70 to 80 percent with the remainder being substantially all helium. The nitrogen concentration in the crude neon 75 product will generally be less than 50 ppm.

The adsorbent bed portion 116 can also contain activated carbon, with molecular sieve occupying the top half of the beds and activated carbon occupying the bottom half of the beds. When catalytic hydrogen removal is carried out as described above, the stream 114 provided into the adsorbent bed portion 116 will additionally contain oxygen and water vapor. The oxygen results from excess oxygen being provided into the catalytic reactor in order to ensure that the hydrogen is completely removed. The water vapor results from incomplete condensation of water vapor in the catalytic reactor effluent. The activated carbon serves to adsorb the water vapor and to chemisorb the oxygen so that the crude neon 75 product contains substantially no oxygen or water vapor.

In addition some oxygen is also adsorbed by the molecular sieve adsorbent. The oxygen concentration in the crude neon 75 product will generally be less than 50 ppm. Conventionally, the resulting crude neon 75 product is then recovered and passed as stream 118 to a neon refinery for the production of product grade neon having a neon purity of 99.99 percent or more. According to embodiments of the present invention however, the crude neon 75 product can instead be used to create breathing mixtures suitable for intermediate pressure ranges (e.g., ˜72 psia) in hyperbaric interventions.

The adsorbent bed portion 116 is desorbed at a pressure less than that at which the adsorption is carried out. Generally, the desorption is carried out at a pressure within the range of from 3 to 14 psia. The ratio of the pressure during the adsorption, or adsorption pressure, to the pressure during the desorption, or desorption pressure, is within the range of from 7 to 20. The low pressure desorption may be carried out by means of a vacuum pump on lines connected to the beds.

The tail gas (i.e., stream 120) resulting from the desorption of the adsorbent bed contains substantially all of the nitrogen which was in the fluid stream 114 provided to the adsorbent bed portion 116. Generally, the nitrogen concentration in the tail gas is within the range of from 40 to 60 percent. The tail gas will also contain some neon, generally at a concentration within the range of from 30 to 50 percent and may also contain oxygen, water vapor and helium. Conventionally, the tail gas is recycled from the adsorbent bed portion 116 back into the cryogenic air separation plant 102. The conventional tail gas recycle to the air separation plant 102 serves to significantly increase the overall neon recovery. However, according to embodiments of the present invention, instead of recycling the tail gas, the stream 120 is recovered for use as crude neon 50 product to create relatively lower cost breathing mixtures suitable for intermediate pressure ranges (e.g., ˜72 psia) in hyperbaric interventions. The crude neon 50 product can be recovered and stored in a container (not shown) for later use. In some embodiments, the crude neon 50 product stream 120 and/or the crude neon 75 product stream 118 can be feed at a metered rate to a mixing chamber (not shown) along with measured amounts of other gases (e.g., N₂, O₂, He, etc.) to form the desired breathing mixtures with the desired proportions of component gases.

Physiologically, neon has two properties that are distinctly advantageous when used in breathing mixtures to divers and hyperbaric workers. First, neon has lower thermal conductivity than other gases. As a result, diver heat loss is reduced. Second, the speed of sound in neon is less than in other gases such as helium. This results in decreased voice distortion at depth which enables better communication.

The use of air in diving, caisson and tunnel boring operations is limited to pressures up to approximately 165 feet of sea water (fsw) which is approximately 6 atmospheres absolute (ata) or approximately 73 psig. Physiologically nitrogen is narcotic at hyperbaric pressures and becomes more debilitating as its partial pressure is increased. Decompression table developers will typically limit divers and hyperbaric workers to partial pressures of nitrogen (ppN₂) of from approximately 4 ata to 5 approximately ata. Therefore nitrogen was conventionally removed from breathing gas mixtures to avoid narcosis in divers, caisson workers and those operators employed in the hyperbaric intervention during tunnel boring. However, partial pressures of less than approximately 4 ata of nitrogen are tolerable and to some degree preferred by divers over pure helium/oxygen mixes due to the lessening of the effects mention above. As mentioned above, these mixtures are known as trimix and for certain decompression modeling programs, the use of trimix has shown reductions of total decompression times.

A disadvantage of neon is that it is denser than helium. At pressures beyond 20 atmospheres, neon has been shown to restrict the ease of breathing when inadequate supply is delivered to the diver/worker's breathing equipment. At pressures beyond about 25 atmospheres (800 fsw), the human ventilator system becomes limiting when breathing neon. This density restriction is not a problem at depths of less than 600 fsw when breathing equipment and critical orifices within the breathing gas supply and the equipment are adequate.

An additional physiological concern about neon is its effect on decompression. Having theoretical compartment halftime solubility and a solubility ratio (oil/water) similar to helium and a diffusivity resembling that of nitrogen, the decompression results predicted for neon would depend largely on which decompression model is considered and the time-pressure profile of exposure. During the 1960s to mid-1970s the Ocean Systems Laboratory at Tonawanda, N.Y. and at Tarrytown, N.Y. developed a model that is tissue compartment perfusion limiting (see H. R. Schreiner and P. L. Kelley, A Pragmatic View of Decompression, in C. J. Lambertsen, Ed., Underwater Physiology IV, Academic Press, New York, 1971 which is hereby incorporated herein by reference). The model and the parameters developed for most suitable inert gases for both hyperbaric and hypobaric exposures evolved over the years into a well-known decompression computational system called DCAP (Hamilton, R. W. et al., which is hereby incorporated herein by reference). Decompression tables designed using DCAP software show relatively little difference from the use of helium to that of neon and crude neon mixtures as the inert diluent. This has a significant advantage in that a mixture ratio of helium to neon is not critical to decompression and larger mixture tolerances can offer a significant economic benefit to the cost of these special breathing mixtures.

Careful oxygen management during a hyperbaric exposure can play and important role in optimizing decompression times. Commercial and Military diving operators using heliox mixtures have taken advantage of the use of enhanced oxygen while at pressure. During decompression the divers would typically shift to air at a comfortable pressure, and then decompress on air throughout the duration of the decompression. The ppO₂ at pressure is dictated by the breathing equipment used and the length of time during the exposure. Typically the ppO₂ would be from approximately 1.1 atm to approximately 1.4 atm when at pressure. During decompression, the oxygen concentration within a fixed volume remains the same. If no additional oxygen is added the ppO₂ will drop proportionally with the pressure, therefore a significant decompression advantage is obtained if oxygen is continuously added during decompression to arrive at an optimal ppO₂ for the duration of the decompression. Again, the ppO₂ is carefully adjusted based on the breathing equipment used and the duration of the decompression times that are required with the decompression tables provided for the return from the pressure exposure. Typically the ppO₂ would be above 1.0 atm. The exposure guidelines technical divers use to manage oxygen toxicity are derived from those published in the NOAA Diving Manual, which is hereby incorporated herein by reference.

Some applications of the present invention include deep diving where crude neon can be used as a replacement for helium in depths from 150 to 600 fsw; caisson construction when pressures exceed approximately 72 psia (165 fsw or 5 atm); tunnel boring when pressures exceed approximately 72 psia; and submarine evacuation when the depth exceeds approximately 150 fsw.

Compressed air work is a significant category of workplace exposure to hyperbaric pressure. When underground construction projects are performed below the water table, the working space may be pressurized with compressed air to keep the water out of the work area. The workers, sometimes called “sand hogs”, are exposed to this pressure for most of each work shift, and of course, they have to decompress at the end of the day. The nature of the work varies but this aspect applies in about the same way to most situations. Working in compressed air is not so much practiced now as in the past, but it has been a major source of injury and death.

Although properly called compressed air work, recent developments that call for work at pressure too great for air breathing are now being done by using breathing mixtures containing helium. According to embodiments of the present invention, lower cost crude neon mixtures can replace the need for heliox mixtures. Compressed air work falls into two major categories: caissons and tunnels. In both cases, people pass into and out of the work areas though pressure locks.

Caissons are usually dug vertically into the ground and are used for structures such as bridge abutments or to support buildings. Except when the underground conditions involve solid reliable rock, it is often necessary to dig down to a rock base or deep enough to provide a good footing for piles to support the structure. As the caisson is dug deeper, a precast structure may be lowered, adding concrete at the top.

Caissons may be dug down in a vertical direction, but tunnels are usually horizontal. Tunnels may be used for roads, railroads, or subways, or for water, sewage, or other underground utilities. Today's tunnels are dug by tunnel boring machines (TBM), large machines that have a toothed “face” that rotates as the machine is forced along through whatever material is present where the tunnel is to go. In favorable rock formations it may not be necessary to use compressed air to keep the water out. The space in front of the “face” may be the only pressurized area; workers may need to work at that pressure. Often the entire process is automated, and the only intervention of workers is to deal with repairs, maintenance and boring “teeth” replacements. As tunnels continue to be built deeper, e.g., at pressures beyond 73 psig (i.e., the effective pressure limit for air) alternative, mixed gas breathing systems are needed for workers operating at advance pressures.

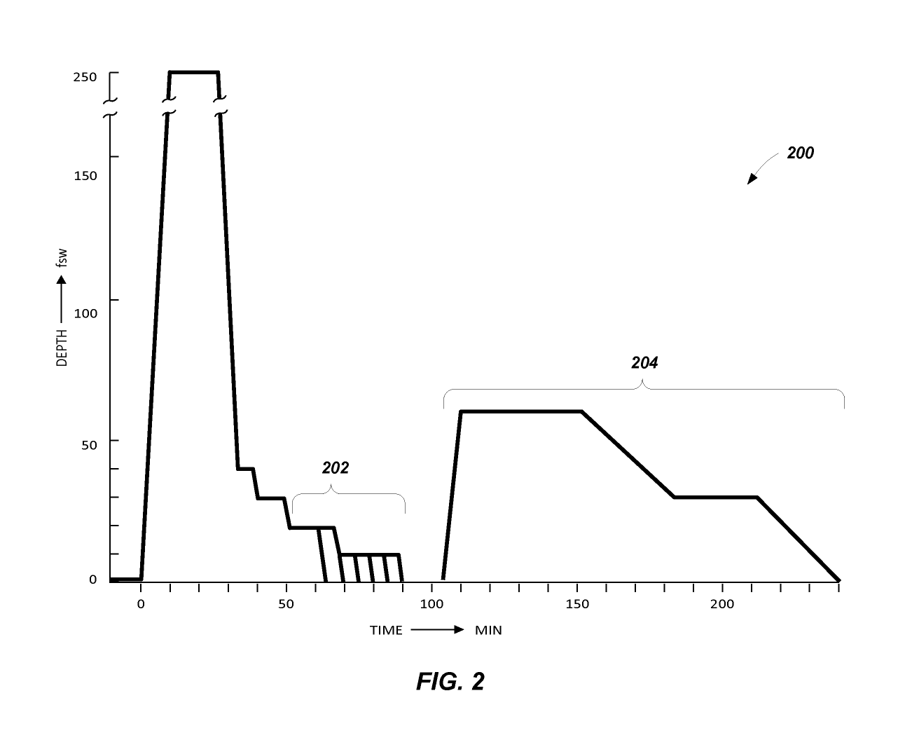

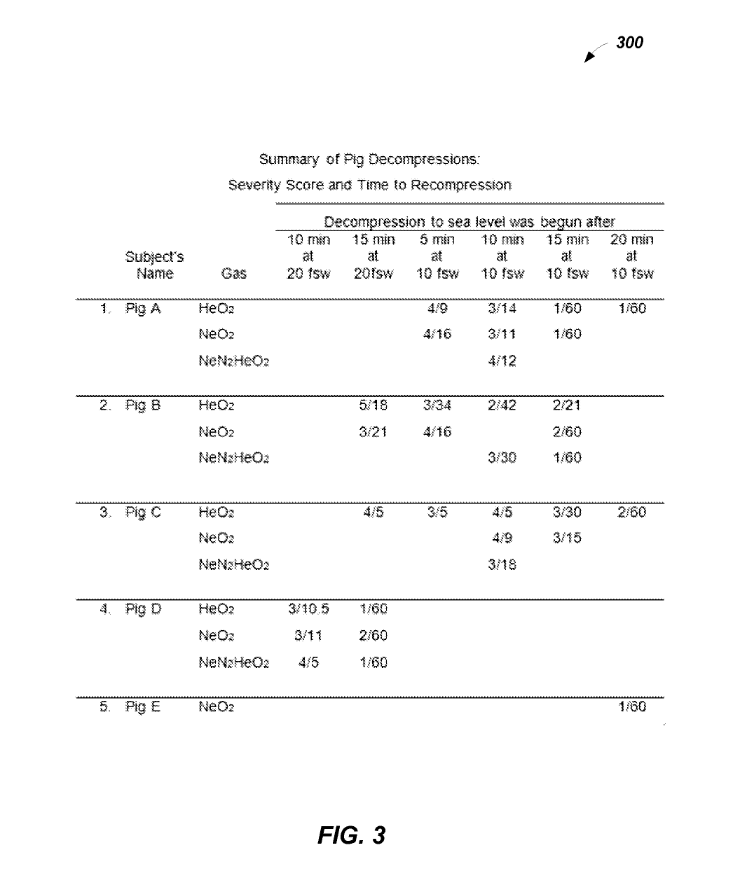

Turning now to FIG. 2, an example dive schedule for pigs breathing helium or neon with oxygen is depicted. Points at which the decompression time was shortened are indicated at the 20 and 10 fsw levels 202. A treatment schedule 204 is also depicted in FIG. 2. Note that treatment does not always commence at 99 minutes but follows the observance of suitable signs of decompression sickness. FIG. 3 is a tabular summary of the decompression results of the example dive schedule shown in FIG. 2 for five different pig test subjects for the different fsw levels 202 of FIG. 2. In table 300 of FIG. 3, the bends score (or severity) is shown as the numerator and the denominator indicates the time elapsed until the development of the sign which resulted in recompression. The severity codes are: 1. No symptoms; 2. Marginal problems, slight reluctance to lift a leg on a treadmill; 3. Reluctance to lift legs when walking, symptoms are definite; 4. Difficulty in walking; drags foot; and 5. Difficulty in walking, slips off the treadmill; often seen to sway back and forth. The fact that almost without exception a shorter table causes a higher score helps to establish the capability of this method for scoring. Additional exposures to both crude neon 75 and Crude Neon 50 were carried out and were compatible with this preliminary data. It can be shown that crude neon mixtures with helium and oxygen or crude neon mixtures with neon, helium, nitrogen and oxygen behave similarly in a decompression sense, to that of helium only mixtures with oxygen.

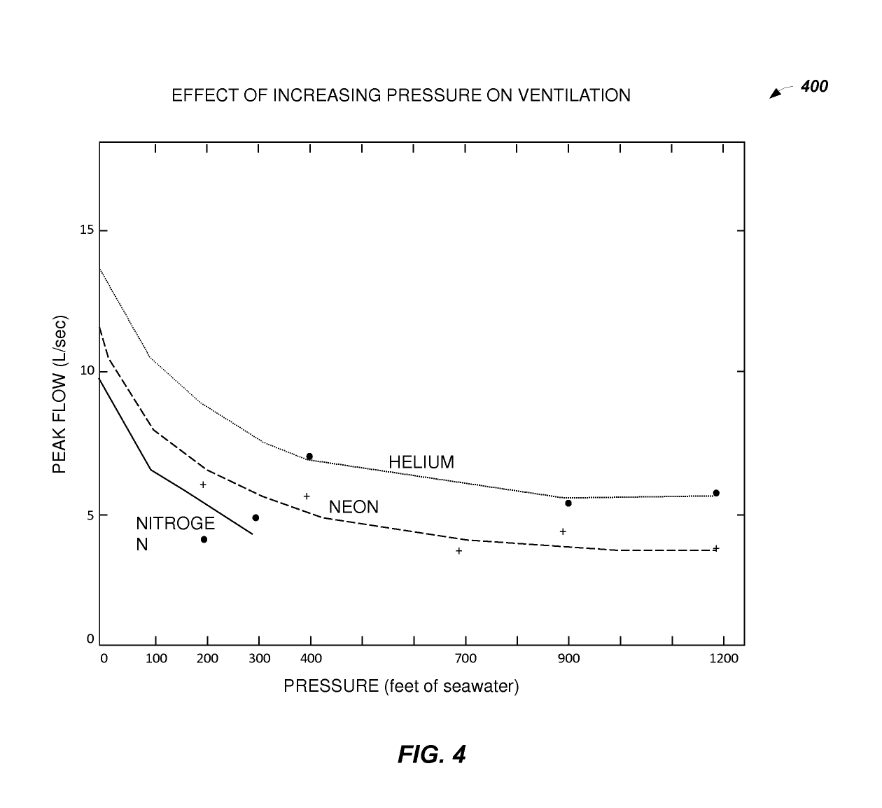

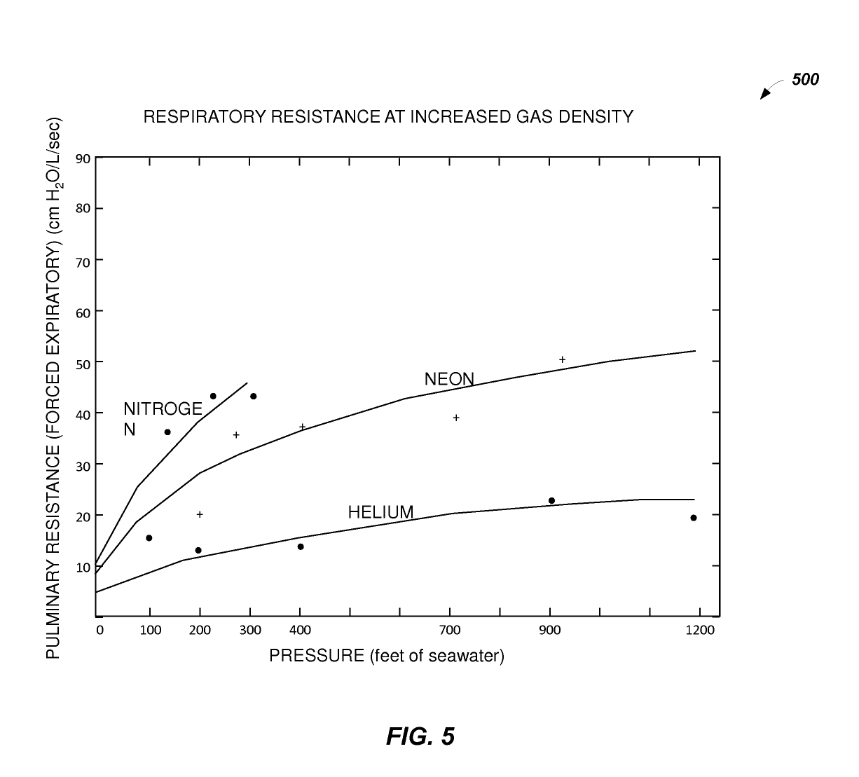

FIG. 4 illustrates that the peak flow of neon changes with pressure in a manner that parallels that of helium. FIG. 5 illustrates that the respiratory resistance at increasing gas densities for neon again parallels that of helium. The graph 400 in FIG. 4 shows that neon as an inert gas diluent in breathing mixtures behaves like helium. Neon is less restrictive within a breathing system than nitrogen (as in the mixture AIR) but more restrictive that helium only mixtures. Because of this additional restriction, neon itself in breathing mixtures, is limited to depths of up to approximately 500 fsw, where crude neon mixtures (with added helium) would be beneficial for increased ventilation and reduced respiratory resistance (as shown in graph 500 of FIG. 5) over pure neon oxygen mixtures.

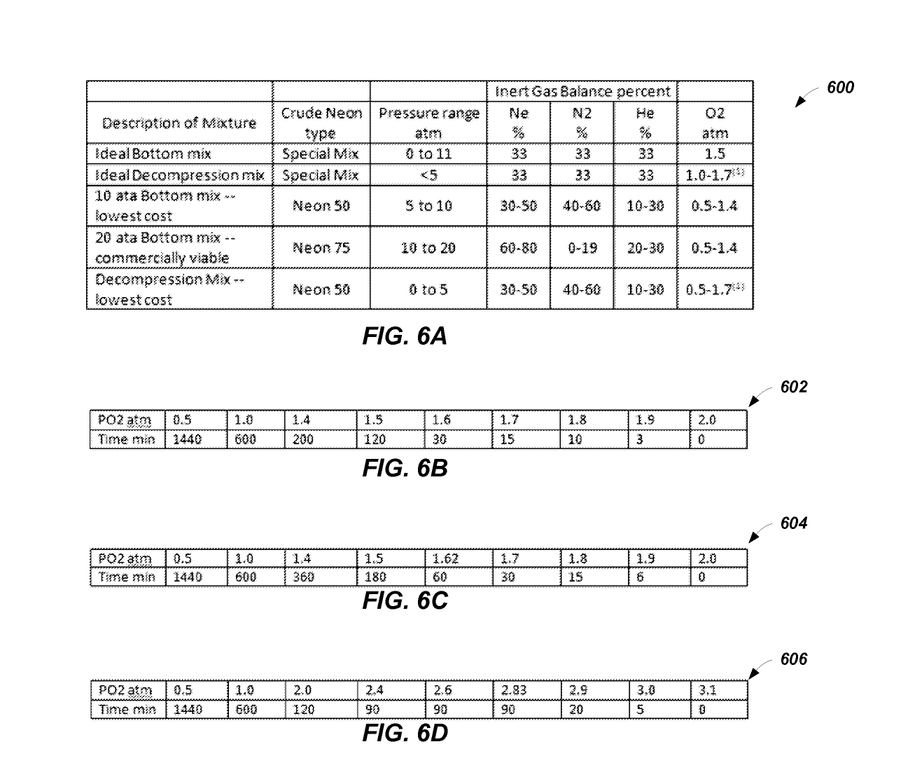

Turning now to FIGS. 6A to 6D, characteristics of example breathing mixtures that include crude neon are presented in tabular form. Table 600 of FIG. 6A lists five example crude neon breathing mixtures and the ranges of the gas component percentages. Table 600 defines acceptable ranges for inert gas component percentages (not including the addition of the necessary Oxygen). As can be seen on the first two lines of table 600, the “ideal” crude neon breathing mixture is a balance of ⅓ Ne, ⅓ He and ⅓ Nitrogen. Note (1) is to point out that 1.7 atm of O₂ can only be tolerated for brief stops and that decompression efficiency is reduced with a ppO₂ of less than 1.0 atm. The amount of oxygen is governed by the central nervous system (CNS) toxicity of oxygen and can be defined by the limits in tables 602, 604, 606 of FIGS. 6B-6D. Table 602 provides time limits for working levels of various oxygen partial pressures, table 604 provides time limits for resting levels of various oxygen partial pressures, and table 606 provides time limits for decompression chamber levels of various oxygen partial pressures. Tables 602, 604 and 606 show the allowed time (bottom row of each table) that can be spent breathing mixtures with oxygen that will result in an oxygen partial pressure listed in the top row. If the time at a particular ppO₂ is exceeded either the diver/subject should be decompressed to achieve a lower ppO₂, or should shift to a mixture with a lower concentration of oxygen. Typically, breathing regimes incorporating cycles of high ppO₂ mixtures and low ppO₂ mixtures. An example would be 25 min on oxygen during chamber decompression and 5 min of AIR (off oxygen). This cycle regime reduces the exposure to the high ppO₂ mixtures, and in some complex decompression computation systems will give credit for divers/subjects going lower than 0.5 atm ppO₂.

The lowest cost breathing mixture (e.g., the most economical to produce) is a mixture created from crude neon 50 that can be directly used from, for example, the output of adsorbent bed portion 116 of the crude neon production system 100 depicted in FIG. 1 and only oxygen is added to make the final breathing mixture.

For re-breather applications there are generally two gas tanks within the system. One tank holds pure (i.e., 100%) oxygen and the second tank holds a low oxygen diluent which does not necessarily contain oxygen. This provides a safer situation for the user/worker in the event that the diluent escapes the second tank and completely fills the counter lung within the breathing apparatus. In some embodiments, the present invention provides a re-breather including a loop blower with high flow and up to 2 psi pressures; a CO₂ absorber such as SodaSorb, Lithium Hydroxide, etc.; a pure oxygen addition from s high pressure cylinder or surface supplied O₂ line; a low percentage oxygen (e.g., 10% +/−2%) breathing mixture with balanced N₂, He and Ne mixture addition from a high pressure cylinder or surface supplied line; optional heating/cooling facilities; and a concentric hose system with quick disconnect. In some embodiments, the present invention provides methods of use of a constant ppO₂ Re-breather. According to such methods, the total decompression time is optimized by maintaining a constant ppO₂ during decompression. The method includes the use of a closed-circuit, mixed-gas, system with a 6 to 12 hour duration, a 500 fsw capability, real-time DCAP software for managing the breathing mixture, an ability to maintain a ppO₂ between 1.1 to 1.3 based on mission/task time, and an adjustment capability to lower the ppO2 to 1.0 throughout decompression up to 100% O₂ breathing less than 40 fsw (18 psi, 1.2 Atm or 12 msw). Further, the method includes the use of N₂, He and Ne in a balanced ratio of N₂ 30% +/−5%, He 30% +/−5%, and Ne 30% +/−5%.

In some embodiments, the present invention provides that an optimized crude neon tail end flow from a cryogenic air reduction gas plant can be a cost effective means to create a balanced breathing mixture of N₂, He and Ne and even O₂. In addition, embodiments of the present invention provide that the crude neon manufacturing process can be optimized for cost to provide all components of a four gas breathing mixture (e.g., a quadmix) with minimal component addition. A final balanced breathing mixture of O₂, and N₂ with crude neon is based on the application and the pressure of the exposure. Furthermore, the ratio tolerance of neon to helium can be wide without compromising the safety of the workers during decompression.

The embodiments further provide that overall decompression time can be optimized using balanced crude neon mixtures and incorporating the use of a breathing apparatus that can deliver a constant ppO₂ such as a re-breather or by changing gas mixtures with enhanced oxygen content throughout the decompression.

In some embodiments, overall decompression time is optimized by the use of a re-breather incorporating a balanced inert gas mixture and a constant ppO2. In some embodiments, the DCAP software is adapted to include decompression tables for the use of crude neon 50 and 75 in a balanced mixture of N₂, He and Ne with a constant ppO₂ during decompression. According to some embodiments, an optimized crude neon tail end flow from a cryogenic air reduction gas plant can be cost effective produced containing a balanced breathing mixture of N₂, He and Ne and, in some embodiments, even O₂. This process can be optimized for cost to provide all components of a four gas mixture (e.g., a quadmix) with minimal component addition for a final mixture of 10%, 30%, 30% and 30% for O₂, N₂, He and Ne respectively.

In some embodiments, the present invention includes DCAP software that is a comprehensive hypobaric/hyperbaric modeling and programming tool that provides decompression profile development, profile analysis, and decompression table publishing that can be used by engineers, physiologists, researchers, dive operations personnel, and medical personnel without the need to write programming code.

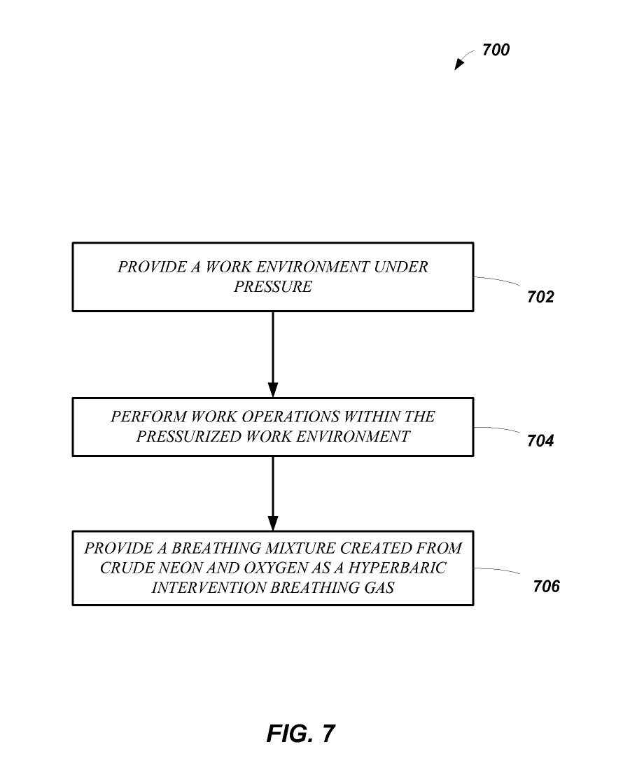

Turning now to FIG. 7, a flowchart depicting an example method 700 of embodiments of the present invention is provided. The method 700 includes providing a work environment under pressure (702). The work environment can be a personal space, an enclosed tunnel section below sea water, an enclosed hole for a caisson, or any other pressurized space for one or more workers. The work environment can be under pressure greater than 72 psia. Work operations are performed within the pressurized work environment (704). To avoid oxygen toxicity and nitrogen narcosis, a breathing mixture created from crude neon and oxygen as a hyperbaric intervention breathing gas is provided to the workers within the pressurized work environment (706). The crude neon can include crude neon 50, crude neon 75, or a combination of the two.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicant intends to file additional applications to pursue patents for subject matter that has been disclosed and enabled but not claimed in the present application. Accordingly, while the invention has been disclosed in connection with example embodiments thereof, it should be understood that other embodiments may fall within the scope of the invention, as defined by the following claims.

What is claimed is:

1. A method comprising:
providing a work environment under pressure;
performing work operations within the pressurized work environment; and
providing a breathing mixture created from crude neon and oxygen as a hyperbaric intervention breathing gas.

2. The method of claim 1 wherein providing a work environment under pressure includes providing a work environment under pressure greater than 72 psia.

3. The method of claim 1 wherein providing a breathing mixture includes providing a breathing mixture that includes crude neon 50.

4. The method of claim 1 wherein providing a breathing mixture includes providing a breathing mixture that includes crude neon 75.

5. The method of claim 1 wherein providing a breathing mixture includes providing a breathing mixture that includes additional gases including nitrogen and helium.

6. The method of claim 1 wherein providing a breathing mixture includes providing a breathing mixture that includes 50% to 60% neon, 20% to 30% nitrogen, and 10% to 30% helium.

7. The method of claim 1 wherein providing a breathing mixture includes providing a breathing mixture that includes 60% to 80% neon, 0% to 19% nitrogen, and 10% to 30% helium.

8. A breathing mixture production system comprising:
an air separation plant;
a hydrogen removal portion configured to receive a first fluid stream from the air separation plant; and
an adsorbent bed portion configured to receive a second fluid stream from the hydrogen removal portion and further adapted to provide crude neon for use in a hyperbaric intervention breathing mixture.

9. The breathing mixture production system of claim 8 further comprising an oxygen supply adapted to provide oxygen to the hyperbaric intervention breathing mixture.

10. The breathing mixture production system of claim 8 further comprising a container to store the hyperbaric intervention breathing mixture coupled to the adsorbent bed portion.

11. The breathing mixture production system of claim 8 wherein the adsorbent bed portion is configured to produce crude neon 50 for use in a hyperbaric intervention breathing mixture.

12. The breathing mixture production system of claim 8 wherein the adsorbent bed portion is configured to produce crude neon 75 for use in a hyperbaric intervention breathing mixture.

13. A method comprising:
providing a decompression chamber under pressure;
decompressing a user within the pressurized decompression chamber; and
providing a breathing mixture created from crude neon and oxygen as a hyperbaric intervention breathing gas.

14. The method of claim 13 wherein providing a decompression chamber under pressure includes providing a decompression chamber under pressure greater than 72 psia.

15. The method of claim 13 wherein decompressing a user includes maintaining a fixed oxygen concentration within the decompression chamber.

16. The method of claim 13 wherein providing a breathing mixture includes providing a breathing mixture that includes crude neon 50.

17. The method of claim 13 wherein providing a breathing mixture includes providing a breathing mixture that includes crude neon 75.

18. The method of claim 13 wherein providing a breathing mixture includes providing a breathing mixture that includes additional gases including nitrogen and helium.

19. The method of claim 13 wherein providing a breathing mixture includes providing a breathing mixture that includes 50% to 60% neon, 20% to 30% nitrogen, and 10% to 30% helium.

20. The method of claim 1 wherein providing a breathing mixture includes providing a breathing mixture that includes 60% to 80% neon, 0% to 19% nitrogen, and 10% to 30% helium.
